(19) United States
(12) Patent Application Publication
(10) Pub. No.: US 2008/0206339 A1
(43) Pub. Date: Aug. 28, 2008

(54) VERY-PURE SUPERPOROUS HYDROGELS HAVING OUTSTANDING SWELLING PROPERTIES

(75) Inventors: Hossein Omidian, Weston, FL (US); Cristian Gavrilas, Hallandale, FL (US); Wenli Han, Long Grove, IL (US); Ge Li, Weston, FL (US); Jose G. Rocca, Miami, FL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/036,680

(22) Filed: Feb. 25, 2008

Related U.S. Application Data
(60) Provisional application No. 60/903,895, filed on Feb. 28, 2007.

(51) Int. Cl. A61K 9/14; A61P 3/04

(52) U.S. Cl. 424/487

(57) ABSTRACT

The invention relates to methods for preparing superporous hydrogels (SPH) that are very-pure and have desirable swelling properties necessary for commercial use, such as in food and pharmaceutical applications. Such methods include simultaneous use of low and high glass transition monomers to improve impurity and swelling profiles of the SPH, use of an integration means to prepare a very homogenous superporous hydrogel foam, washing the superporous hydrogel in a washing solution comprising different ratios of solvent to non-solvent (e.g., water/alcohol), use of a chemically-induced expansion/contraction process to enhance the efficiency of the multiple washing process and to fully structuralize the SPH, and employing one or more separation techniques, such as rubbing, filtration, centrifugation, compression and cutting to increase the efficiency of the purification process and to enhance the SPH swelling properties.

### CROSS-REFERENCE SECTION TO RELATED APPLICATIONS

This application claims priority to U.S. Application Ser. No. 60/903,895, filed Feb. 28, 2007, and is incorporated herein by reference in its entirety.

### FIELD OF THE INVENTION

The present invention relates to polymers and more specifically methods of producing very-pure superporous hydrogels having outstanding swelling properties.

### BACKGROUND OF THE INVENTION

Superporous hydrogels (SPHs) are chemically crosslinked hydrophilic polymers that contain pores with diameters in the micrometer to millimeter range, enabling them to absorb tens times their own weight of aqueous fluids in a fraction of minute. SPH pores are interconnected in the hydrogel matrix such that absorbing fluid can move freely through the channels (capillaries), allowing SPH to swell much faster than conventional hydrogels that have the same swelling capacity. The amount of the fluid absorbed within the SPH structure depends on the thermodynamic swelling capacity of the gel and also the swelling conditions.

To prepare a superporous hydrogel, monomer(s), a crosslinker, a solvent (also known as a dilutent, e.g., water), a surfactant (for foam stabilization), and a foaming aid (typically an acid) are first mixed together, followed by the addition of an initiator (typically two components, such as an oxidant and reductant). A foaming agent (also commonly known as a blowing agent) is then added to the mixture for the generation of gas. i.e., carbon dioxide via reaction with the acid. Once the initiator and foaming agent are added, foaming and polymerization (also referred as gelling) processes take place simultaneously. As polymerization proceeds, the viscosity of the reaction mixture increases and the generated gases are trapped within the highly viscous polymer matrix. The foaming resulting from simultaneous gelation and gas formation continues until both processes are completed. At this stage, the product resembles flexible foam. The foam further undergoes washing, purification, and drying processes in order to be exploited as a swelling agent.

With respect to purification, techniques known to those skilled in the art give rise to three general classes of impurities in the final SPH product: (i) synthetic impurities, (ii) washing impurities, and (iii) drying impurities. Synthetic impurities are generally classified as unreacted monomers that remain in the SPH pores and gel structure. Washing impurities are residual solvents that remain in the SPH subsequent to the washing of the SPH. Lastly, drying impurities are residual chemicals that remain from the drying process, e.g., a dehydrating solvent such as alcohol.

Due to the impurities produced when making the final SPH product, there is a need for an improved method of preparing a SPH that has reduced impurity content and adequate commercial applicability.

### SUMMARY OF THE INVENTION

The present invention provides a method for preparing a superporous hydrogel (SPH) that is very-pure and has desirable swelling properties necessary for commercial use, such as in food and pharmaceutical applications. Generally, in one embodiment, the invention provides a method of producing a very-pure superporous hydrogel including the steps of:
a. preparing a superporous hydrogel reacting mixture including a high-glass transition temperature (Tg) ethylenically unsaturated monomer (HG) and a low-glass transition temperature (Tg) ethylenically unsaturated monomer (LG);
b. evenly integrating a foaming agent into the superporous hydrogel reacting mixture of step (a) using an integration mechanism to create a homogeneous mixture of the foaming agent within the superporous hydrogel reacting mixture;
c. washing the superporous hydrogel foam of step (b) one or more times in a washing solution including a solvent and a non-solvent to remove impurities from the hydrogel of step (b); and
d. drying the washed superporous hydrogel of step (c) to form a very-pure superporous hydrogel.

### BRIEF DESCRIPTION OF THE DRAWINGS

FIG. 1 is the swelling profile of a sulfopropyl acrylate-based (HG) SPH in an ethanol/water mix.

FIG. 2 is the swelling profile of a sulfopropyl acrylate/acrylate ester-based (combined HG-LG) SPH in an ethanol/water mix.

FIG. 3 shows region 3 of the FIG. 2, which graphs the swelling profile of a sulfopropyl acrylate/acrylate ester-based SPH in an ethanol/water mix at very high alcohol concentration (80 to 100 v/v % ethanol).

### DETAILED DESCRIPTION

The present invention relates to methods for preparing superporous hydrogels (SPH) that are very pure and have desirable swelling properties necessary for commercial use, such as in food and pharmaceutical applications. Such methods include simultaneous use of low and high glass transition monomers to improve impurity and swelling profiles of the SPH, use of an integration means to prepare a very homogenous superporous hydrogel foam, washing the superporous hydrogel in a washing solution including different ratios of solvent to non-solvent (e.g. water/alcohol), use of a chemically-induced expansion/contraction process to enhance the efficiency of the multiple washing process and to fully structuralize the SPH, and employing one or more separation techniques, such as rubbing, filtration, centrifugation, compression and cutting to increase the efficiency of the purification process and to enhance the SPH swelling properties.

As used herein, the term, “high-glass transition ethylenically-unsaturated monomer,” refers to monomers carrying one or more unsaturated bonds, wherein their corresponding polymers have a glass transition temperature above room temperature.

As used herein, the term, “low-glass transition ethylenically-unsaturated monomer,” refers to monomers carrying one or more unsaturated bonds, wherein their corresponding polymers have a glass transition temperature below room temperature.

As used herein, the term, “very-pure,” refers to a superporous hydrogel having synthetic impurities, washing impurities, and drying impurities less than 25 ppm and 40 ppm respectively, and preferably 10 ppm to 20 ppm.

As used herein, the term, “impurities,” refers to (i) synthetic impurities generally classified as unreacted monomers that remain in the SFH pores and gel structure, (ii) washing impurities generally classified as residual solvents that remain in the SPH subsequent to the washing of the SPH, and (iii) drying impurities generally classified as residual chemicals that remain from the drying process, e.g., a dehydrating solvent such as alcohol.

Generally, in one embodiment, the invention provides a method of producing a very-pure superporous hydrogel including the steps of:
e. preparing a superporous hydrogel reacting mixture including a high-glass transition temperature (Tg) ethylenically unsaturated monomer (HG) and a low-glass transition temperature (Tg) ethylenically unsaturated monomer (LG);
f. evenly integrating a foaming agent into the superporous hydrogel reacting mixture of step (a) using an integration mechanism to create a homogeneous mixture of the foaming agent within the superporous hydrogel reacting mixture;
g. washing the superporous hydrogel foam of step (b) one or more times in a washing solution including a solvent and a non-solvent to remove impurities from the hydrogel of step (b); and
h. drying the washed superporous hydrogel of step (c) to form a very-pure superporous hydrogel.

The combination of both low and high glass transition monomers in the superporous hydrogel formulation increases the efficiency of the purification process. In one embodiment of the invention, the flexibility of the SPH network during the washing and drying steps serves as an influential factor in the purification process. Increased flexibility of the SPH network in the washing solution results in a faster and more efficient removal of unwanted chemicals (i.e., residual, unreacted starting materials from SPH synthesis, washing and drying steps). Desirable flexibility can be achieved by changing concentrations of the low (e.g., acrylate ester) and high (e.g., acrylate salt) glass transition monomers. An SPH network richer in acrylate and acrylate salt monomer will be structurally more flexile and rigid respectively. In a further embodiment of the invention, the foaming agent is a solid bicarbonate powder and the integration mechanism is a powder gun that is employed to evenly disperse the bicarbonate powder within the previously prepared superporous hydrogel reacting mixture.

The purification method of the invention can further include multiple washing steps applied to the superporous hydrogel, as described in step (c) above, wherein each wash utilizes a washing solution including different ratios of solvent to non-solvent (e.g., water/alcohol). For example, in one embodiment, a multiple washing step method can include the steps of, i) washing the superporous hydrogel in mixed water/alcohol solution having a low alcohol content, ii) washing the superporous hydrogel of step (i) in mixed water/alcohol solution having a medium alcohol content, and iii) washing the superporous hydrogel of step (ii) in mixed water/alcohol solution having a high alcohol content. Preferably, after completion of a washing step, the washing solution is removed from the superporous hydrogel, prior to initiating another wash. In this way, a fresh washing solution is utilized for each washing step.

A further embodiment of the invention employs a chemical expansion/contraction process to enhance and increase the efficiency of a multiple washing process as described herein. One can induce contraction and expansion by submerging the SPH in solutions rich in non-solvent and solvent respectively. In this way, controlled expansion and contraction between each washing step serve to further purify and structuralize (opening of the cellular structure of an SPH) the SPH. In one embodiment, a substantially pure alcohol solution is used subsequent to each washing step (for example, washing steps (i) to (iii) above). In this way, one can induce SPH contraction after each individual washing step prior to beginning the next wash. Expansion of the SPH during a washing step can be controlled by the concentrations ratio of solvent (i.e., water to be absorbed by the SPH) in the washing solution.

In a further embodiment, one or more separation techniques, such as rubbing, filtration, centrifugation, compression and cutting can be utilized to increase the efficiency of the purification process and to enhance the SPH swelling properties. Separation techniques can be employed between each washing step, or after completion of the washing process, but are generally employed prior to the drying step. Using separation techniques after each wash, such as centrifugation facilitates the separation of impurities from the SPH structure and also helps to more adequately remove residual solvent (containing impurities) from the washing process prior to initiating another wash. In this way, separation techniques prevent carrying over impurities from one washing medium to the next. Accordingly, separation techniques employed between washing steps can minimize the number of washing steps necessary to reach desired purification.

To prepare the superporous hydrogels of the present invention, low and high glass transition ethylenically-unsaturated monomer(s) are mixed with several ingredients in a polymerization reaction. The mixture can include one or more co-monomers, crosslinkers, diluents, foaming aids, foaming stabilizers, initiators, and foaming agents. The mixture can be polymerized by any method known to those skilled in the art. Polymerization techniques can include, for example, solution, suspension, microsuspension, inverse suspension, dispersion, emulsion, microemulsion, and inverse emulsion polymerization. Methods for synthesis of SPH are described generally in U.S. Pat. No. 6,271,278 and Chen, et al., in J. Biomed. Mater. Res. 44:53-62 (1999), all of which are incorporated by reference.

The low and high glass transition ethylenically-unsaturated monomer of the present invention can be any monomer known to those skilled in the art demonstrating favorable properties (e.g., swelling and gelling). Examples of suitable high glass transition ethylenically unsaturated monomers (HG) include, but are not limited to, acrylic acid (AA), methacrylic acid, (meth)acrylic salts, acrylamide (AM), N-isopropylacrylamide (NIPAM), methacrylamide, itaconic acid, potassium 3-sulfopropyl acrylate (SPAK), potassium 3-sulfopropyl methacrylate (SPMAK), hydroxyethyl methacrylate (HEMA), hydroxypropyl methacrylate (HPMA), methyl methacrylate, stearyl methacrylate, and any other suitable high glass transition ethylenically unsaturated monomers (HG) known to those of skill in the art.

Examples of suitable low glass transition ethylenically unsaturated monomers (LG) include, but are not limited to N,N-dimethylaminoethyl acrylate, 2-hydroxyethyl acrylate (HEA), 2-hydroxypropyl acrylate (HPA), butanediol monoacrylate, ethyl acrylate, butyl acrylate, butyl methacrylate, isodecyl methacrylate, lauryl methacrylate, and any other suitable low glass transition ethylenically unsaturated monomers (LG) known to those of skill in the art.

Preferably, the reaction mixture includes an acrylate ester (LG monomer) as a primary monomer and an acrylate salt (HG monomer) as a co-monomer. The ratio of low and high glass transition monomer utilized in the SPH foam can vary depending upon the desired properties of the final SPH product.

Although wide ratios of low to high glass transition monomers can be used based on the properties desired by one skilled in the art for the final SPH, one embodiment of the present invention employs a ratio of the one or more low T_{g} ethylenically unsaturated monomers to the one or more high T_{g} ethylenically unsaturated monomers of about 1:10 to 10:1. A further embodiment employs a weight ratio of about 2 to 3 grams of low T_{g} monomer to 1 gram of high T_{g} monomer.

The crosslinking agent of the present invention can be any agent known to those skilled in the art. Examples of suitable crosslinking agents include, but are not limited to, glutaraldehyde, epichlorohydrin, degradable crosslinking agents such as crosslinkers containing 1,2-diol structures (e.g., N,N′-diallyltartardiamide and ethylene glycol dimethacrylate), functionalized peptides and proteins (e.g., albumin modified with vinyl groups), ethylene glycol di(meth)acrylate, trimethylolpropane triacrylate (TMPTA), N,N′-methylenebisacrylamide (BIS), or piperazine diacrylamide, and any other suitable crosslinking agents known to those of skill in the art. Multiolefinic crosslinking agents containing at least two vinyl groups, such as ethylene glycol di(meth)acrylate, poly(ethylene glycol) di(meth)acrylate, trimethylolpropane triacrylate (TMPTA), N,N′-methylenebisacrylamide (BIS), piperazine diacrylamide, crosslinkers containing 1,2-diol structures and two vinyl groups (e.g., N,N′-diallyltartardiamide or ethylene glycol dimethacrylate) are preferred. A preferred crosslinking agent is poly(ethylene glycol) diacrylate. In the present invention, the ratio of crosslinking agent to total monomer is 2-4 wt % (e.g., 2 to 4 grams of crosslinking agent per 100 grams of total monomer).

A foam stabilizer can be used to stabilize the foam until the beginning of the gelling process. Examples of suitable surfactants for use as a foam stabilizer in the present invention include, but are not limited to, Triton surfactants, Tween and Span surfactants, Pluronic® surfactants (poly(ethylene oxide)-poly(propylene oxide)-poly(ethylene oxide) tri-block copolymers) (BASF), Silwet® surfactants (Osi Specialties Inc.), sodium dodecyl sulfate (Bio-Rad Laboratories), albumin (Sigma Chemical Company), gelatin, combinations thereof, and any other similar surfactants known to those of skill in the art. Preferably, Pluronic® F127 (F127) is used as a surfactant.

In the synthesis of a superporous hydrogel by a gas blowing technique, foaming and polymerization have to occur simultaneously, making it important to control the timing of these reactions. The blowing or foaming agent, which is added to the superporous hydrogel reacting mixture of the present invention to prepare a SPH can be, but is not limited to, inorganic foaming agents such as sodium bicarbonate and ammonium bicarbonate, organic foaming agents such as azo compounds such as azodicarbonamide, barium azodicarboxylate and azobisisobutyronitrile, nitroso compounds such as N,N′-dinitrosopentamethylenetetramine and N,N′dinitroso-N,N′-terephthalamide, and hydrazide compounds such as p-toluenesulfonylhydrazide. These foaming agents can be used either individually or in combinations thereof. Sodium bicarbonate (SBC) is a preferred foaming agent.

A foaming aid can be utilized to react with a foaming agent to generate gas (acid/carbonate reaction produces carbon dioxide gas). Generally, suitable foaming aids can be any type of organic acid, including, but not limited to, acrylic acid, acetic acid, or citric acid or any type of inorganic acid including, but not limited to, hydrochloric acid.

Polymerization can be initiated by any polymerization-initiator system, which is suitable for the polymerization of unsaturated monomers in the homogeneous or heterogeneous phase. In general, initiator systems that can be used in the process according to the present invention are known to the person skilled in the art of polymer chemistry. Without restricting the present invention, such initiators are preferably free-radical or free-radical forming compounds or mixtures of substances, such as, for example, hydroperoxides (preferably cumyl hydroperoxide or tert.-butyl hydroperoxide), organic peroxides (preferably dibenzoyl peroxide, dilauryl peroxide, dicumyl peroxide, di-tert.-butyl peroxide, methyl ethyl ketone peroxide, tert.-butylbenzoyl peroxide, diisopropyl peroxydicarbonate, dicyclohexyl peroxydicarbonate, di-tert.-butyl peroxalate, inorganic peroxides, preferably potassium persulfate, potassium peroxydisulfate or hydrogen peroxide), azo compounds (preferably azobis(isobutyronitrile), 1,1′-azobis(1-cyclohexane nitrile), 4,4′-azobis(4-cyanovaleric acid) or triphenyl-methylazobenzene), redox systems (preferably mixtures of peroxides and amines, mixtures of peroxides and reducing agents, optionally in the presence of metal salts) and/or chelating agents. The initiator systems can be pure or in the form of mixtures of two, three or more different initiator systems. In one embodiment of the present invention, a redox pair of an oxidant (e.g., ammonium persulfate) and a reductant (e.g., N,N,N′,N′-tetramethylethylenediamine (TEMED)) is used as an initiator. In a preferred embodiment, a thermally decomposable initiator (such as ammonium persulfate) or a milder redox pair of ammonium persulfate (APS) and bisulfites (for example, sodium metabisulfite) are employed.

The method to wash, purify, and dry a very-pure superporous hydrogel includes the steps of a) removing impurities using a washing solution containing varying ratios (v/v %) of solvent and non-solvent (e.g., water and alcohol) at different water concentrations, and b) removal of water using substantially pure non-solvent solutions, and c) removing alcohol by drying the superporous hydrogel at a pressure of less than 50 Torr or by heat. The non-solvent can include, but is not limited to, methanol, ethanol, 1-propanol, 2-propanol, tetrahydrofuran, dioxane, formic acid, acetic acid, acetonitrile, nitromethane, acetone or 2-butanone. Preferably, the non-solvent is ethanol.

In one embodiment, the method for producing a very-pure superporous hydrogel provides a very-pure superporous hydrogel characterized by a swollen gel to dry gel weight swelling ratio of about 161 to 210 g/g, and a core disappearance time of about 20 to 130 seconds.

In the scope of the present invention, monomers used to prepare a very-pure superporous hydrogel can be selected from monomers having low to high glass transition temperatures. While monomers like acrylic acid, sodium acrylate, potassium acrylate, acrylamide, and sulfopropyl acrylate can result in polymers with high glass transition temperatures, acrylate esters including, but not limited to, hydroxyethyl acrylate, ethyl acrylate, methyl acrylate and similar esters result in polymers with low glass transition temperatures. At a given low-medium drying temperature (30-50° C.), the former and the latter stay respectively rigid and flexible throughout the drying process. Rigidity of the superporous hydrogel prevents the non-solvent (alcohol for example) from being removed effectively from the final product. On the other hand, extreme flexibility can affect and deteriorate the porous structure of the superporous hydrogel, as flexible polymer chains can collapse onto each other. Careful selection of both high and low glass transition monomers provides a superior superporous hydrogel after drying. The following example (Table 1) shows the concentration of the residual alcohol for two SPH formulations synthesized having high (dominant) levels of High and Low T_{g} monomers. The SPH rich in High T_{g} monomer contains almost 1400 times more residual alcohol compared to the SPH rich in Low T_{g} monomer. This significant difference can mainly be accounted for in terms of the structural rigidity and flexibility of the corresponding SPH polymers during drying.

TABLE 1

| | Dominant High Tg | Dominant Low Tg |
|---|---|---|
| | monomer | monomer |
| Analyte | μg/g of dry SPH | μg/g of dry SPH |
| Non-solvent (alcohol) | 46,650 | 33.6 |

Concentration of residual alcohol was determined by a validated Gas Chromatography method and the data represents the microgram (μg) of impurity per gram (g) of SPH. Alternative to this approach will be the application of drying temperature higher than the glass transition temperature of the polymer. This requires using blanket atmosphere like nitrogen as well as vacuum if drying at high to very high temperature is attempted. Since most highly swelling superporous hydrogels are based on ionic monomers with corresponding high glass transition temperatures, a high drying temperature cannot be feasible even if vacuum drying is attempted. For superporous hydrogel compositions containing low glass transition monomers, this technique can feasibly bring required flexibility during the drying process. The drying vessel can be of any size or shape, depending in part on the specific SPH batch size and also in part on the water content of the SPH to be fed to the vessel. The preferred drying vessel is composed of flat screen trays so that the SPH can be spread in monolayer for homogeneous and efficient drying. The air entering the drying vessel can be exchanged in a closed or open loop system. The superporous hydrogel can be dried out using a variety of drying equipment such as, flash dryer, band dryer, plat dryer, rotary dryer, fluid-air dryer, vacuum dryer, tray dryer, spray dryer and freeze dryer. Drying can be conducted at various temperatures and for varying times, but regardless of means, should be employed in a manner to effectively dry the SPH as is known to those skilled in the art. For one embodiment of the processes of the invention, drying is performed in an air forced oven at temperature of 30° C, to 50° C., preferably 40° C. to 50° C. for a period of 8 to 24 hours, preferably 12-18 hours.

Preparation of superporous hydrogels requires addition of a very reactive solid (i.e., the foaming agent) into a liquid reacting mixture (i.e., the SPH reacting mixture). Proper addition of a solid foaming agent is important in SPH formation due to various factors such as the following. There is generally a 5-15 second timeframe for the solid foaming agent to be completely and evenly dispersed into the reacting medium. Further, in a 5-15 second timeframe, dispersion and dissolution of the solid foaming agent take place simultaneously. The solid foaming agent quickly reacts with the acidic foaming aid to produce carbon dioxide gas. The foaming agent in even its solid state is very reactive and reacts easily with the acidic foaming aid in the reacting solution. Additionally, the pH of the reacting medium increases as the foaming aid is consumed in the reaction with the foaming agent. As pH increases, gelation reaction is favored and reacting mixture starts to gel. Viscosity of the reacting foam significantly increases as gelation proceeds. In less than a minute, the reacting mixture turns from a running liquid to a rubbery mass. Any kind of mixing at this step is associated with bubble entrapment inside the reacting mass. Finally, bubbles are normally much bigger than the pores within the SPH and might adversely affect the physical and mechanical properties of the SPH.

Alternative means for adding the solid foaming agent to a SPH foam include, but are not limited to, addition of the solid foaming agent as solution in aqueous and organic/aqueous media; addition of the solid foaming agent as dispersion in organic media like alcohols, glycols, emulsions, suspensions, oils, etc.; the particles of the foaming agent can be coated with water-repellant materials like saturated or unsaturated fatty acids in order to control its dissolution; encapsulate particles of the foaming agent; and addition of a foaming agent as paste, in which monomer can, for, example, be used as the dispersing medium.

The improper addition/dispersion of the solid foaming agent can cause a large variation of foaming agent concentration throughout the polymerizing mixture. This will not only result in non-uniform foam formation, but also significantly impact the polymerization rate due to the local pH variation in the mixture. The formed SPH can contain unwanted heterogeneous layers of hydrogel and superporous hydrogel, pores of broad different sizes and entrapped are bubbles. Further, the final SPH polymers contain higher impurities if the foaming agent is added improperly. Within the scope, of the present invention, the integration means utilized to effectively integrate the foaming agent into the SPH reacting mixture is any system of integration known to those skilled in the art of SPH synthesis. Suitable examples are described herein. In one embodiment of the invention, the foaming agent is added as a solid powder and the integration means includes a two step process wherein the foaming agent is first (i) evenly spread onto the surface of the SPH reacting mixture, and thereafter (ii) homogeneously dispersed through out the SPH reacting mixture. Applicants have found that the use of an effective integration mechanism that provides both efficient spreading and dispersion capable of quickly delivering a fast, homogeneous dispersion of the solid foaming agent particles into the liquid reacting medium is an ideal technique for the preparation of a very-pure SPH. Within the scope of integration mechanism, and example of a suitable spreading technique is a powder gun (such as a compressed air powder coating system), which can be employed to evenly distribute the solid foaming agent particles efficiently upon the surface of the SPH reacting mixture, although any suitable powder coating system can be employed. Examples of a suitable dispersion technique include the use of a high-speed mixer or homogenizer to evenly disperse the solid foaming agent particles on the surface of the SPH reacting mixture homogeneously throughout the mixture. A preferred integration mechanism incorporates a powder gun and a homogenizer.

Although compressed air powder guns used for the efficient delivery of solid particle powders can vary depending on the needs of one skilled in the art, in general, when employing a powder gun, one loads a desirable amount of the solid foaming agent into a tube attached to a pressurized airflow. By controlling the air pressure, the powder can be blown through a wide mouth opening into the polymerization mixture within seconds. The spray pattern can be carefully controlled by a specially designed opening which generates a uniform flow to completely and evenly cover the surface of the reaction mixture and avoids generation of air bubbles. The position of the gun above the reactor can also be taken into consideration. Alternative settings and delivery mechanisms will be known to those skilled in the art and can be adjusted depending upon the spreading technique utilized, particle size and type of solid foaming agent and desired final characteristics of the SPH. For example, applicants found that 13.8 grams of bicarbonate powder can evenly be sprayed into a 190 mm (opening) reactor within 3-4 seconds if 32-35 psi airflow is applied to effectively distribute the bicarbonate powder. With the help of paddle mixing, the sprayed powder was thoroughly dispersed within the SPH reacting mixture. In this way, very homogeneous SPH are formed free from hydrogel layers or spots, air bubbles or areas of unwanted structure.

Particle size of the solid particle foaming agent can impact integration of the foaming agent into the SPH reacting mixture and SPH synthesis. In general, the finer the foaming agent particles, the faster the gelation and foaming reaction. Particle size also affects the SPH porosity. As far as the SPH impurities are concerned, finer particles theoretically result in purer products. On the other hand, since finer particles tend to absorb moisture faster than the larger ones, they might aggregate faster and behave like larger particles. Accordingly, one skilled in the art will take into consideration the particle size of the foaming agent with respect to the integration means employed and the desired final SPH product. In one embodiment, the foaming agent solid particles have a particle size in the range of 10 to 1000 microns, preferably 50 to 200 microns.

With the present invention, a washing solution containing varying ratios (v/v %) of solvent and non-solvent is utilized. Almost all components of a high swelling superporous hydrogels are freely soluble in water. Therefore, water is a preferred solvent to wash the impurities out of the final superporous hydrogel product (other suitable solvents are known to those skilled in the art and can be employed as needed depending upon SPH starting components). On the other hand, water itself is a strong swelling medium for the final product in which the SPH swells to a large swollen mass. Such a large fully swollen mass cannot keep its integrity during washing process and disintegrates to smaller swollen particles. For most applications, the superporous hydrogel is prepared in a special shape (e.g., a cylinder, sheet film, granule, particle, spheroid, cone, cube, rod, or a tube) and the SPH needs to maintain its desired shape even after drying. Using water as the washing medium furthermore requires large equipment and containers for storage and handling of the swollen hydrogel, which is economically undesirable. Adding salts to increase the ionic strength of the swelling medium can help to reduce the size of the swollen hydrogel, but adds other unwanted impurities (salts) into the gel, which can significantly affect the swelling properties of the final product, as well as SPH stability during storage.

Although most of the components of the superporous hydrogel have a certain solubility in alcohol, alcohol alone cannot be utilized to prepare a very-pure superporous hydrogel product. Superporous hydrogels tend to precipitate in alcohol, by which residual impurities are trapped inside the hydrogel structure. Accordingly, an embodiment of the invention employs multiple wash steps using at each step a washing solution containing varying ratios of solvent and non-solvent compositions (preferably water and ethanol respectively in case of high swelling SPH). A suitable composition tends to provide both sufficient solubility and adequate diffusion coefficients for the removal of impurities and desirable mechanical properties for the SPH throughout the washing and purification processes. Suitable solvent/non-solvent ratios can be identified by graphing the SPH swelling versus alcohol concentration for various water/alcohol compositions. In a preferred embodiment a solvent and non-solvent composition characterized by a medium to high solvent content contains greater than 10 v/v % solvent (e.g., water), and a solvent and non-solvent composition characterized by medium to low solvent content contains less than 10 v/v % solvent. Examples 1 and 2 illustrate how the swelling profile of the SPH in solvent/non-solvent compositions can be utilized to prepare a very-pure superporous hydrogel.

The SPHs respond to the swelling media as they swell and de-swell in a solvent and non-solvent respectively. In one embodiment of this invention, frequent cycles of swelling/de-swelling in solvents and non-solvents can be exploited as an additional driving force to remove impurities out of the swollen SPH and to open up the cellular structure of the SPH. The washing/purification process as described above can include several consecutive steps using different solvent/non-solvent (e.g., water/ethanol) solutions in which later steps contain more ethanol than the earlier ones. Although this procedure significantly lowers the level of impurities, the procedure can also inversely affect the swelling properties of the SPH. Therefore, an embodiment of the process of the invention includes incorporating an expansion/contraction mechanism to lower impurity level, as well as increase the swelling rate, of a very-pure SPH. In one embodiment of this invention, after washing an SPH in water/ethanol solution containing low ethanol, one transfers the swollen SPH into a substantially pure solution of non-solvent (e.g. ethanol). As used herein, the term “substantially pure” refers to a solution containing greater than 90% non-solvent, preferably greater than 95%, more preferably greater than 99%. In this way, the swollen SPH contract immediately and hence squeeze the existing washing solution out of the pores. This process can be repeated as necessary to bring the level of impurities to or below an accepted value. Table 2 below illustrates the analytical and swelling data of a single SPH batch, prepared in accordance with the invention (step (a)), separated into two samples that were subjected to different purifying techniques in accordance with the invention (e.g., a single SPH cut into multiple samples prior to purification steps). A validated HPLC method has been used to measure the impurities and the impurity data are in microgram/gram of a dry SPH. The SPH swelling has been measured gravimetrically. Data shows while two samples possess the same amount of impurities, the expansion/contraction of the SPH during washing can significantly help to obtain faster swelling kinetics.

TABLE 2

| | Standard Washing | Expansion/Contraction |
|---|---|---|
| Analyte | μg/g of dry SPH | μg/g of dry SPH |
| Acrylic monomer | <2.62 | <2.62 |
| Acrylate ester monomer | <24.3 | <24.3 |
| Acrylate salt monomer | <2.46 | <2.46 |
| Stabilizer | <25.3 | <25.3 |
| Diacrylate Crosslinker | <9.48 | <9.48 |
| Rate Swelling* | >4 min | 100-120 sec |

*Time by which the SPH reaches its full swelling capacity

A further embodiment of the invention includes the techniques to achieve superior desirable impurity and swelling profiles. These separation techniques include, but are not limited to, rubbing, filtration, centrifugation, compression and cutting. Generally, a SPH is transferred to the washing solution containing alcohol and water after the SPH is synthesized. In general, gels expand more if they are rubbed in the washing solution. The rubbing process helps to open up the microporous structure of the SPH by which more solution can enter the hydrogel network, thereby increasing swelling capacity. In addition, rubbing can increase the efficiency of the washing and purification processes. In one embodiment of the invention, after synthesis, the SPH will be placed in a tank containing the washing solution. The swollen gels are rubbed using a sufficient rubbing means, (e.g., a paint roller) to the point that all swollen SPH reach an equivalent size and transparency.

To further remove the impurities and/or the residual (absorbed) washing medium inside the SPH, gravity decantation devices, screen filter, batch filter, centrifuges and vacuum devices can be used. In one embodiment of this invention, a mechanical centrifuge is loaded with the swollen SPH. In this way, the absorbed washing solution can efficiently be removed from the SPH. The centrifuged SPH is fluffy foam having a very open cellular structure. The centrifuge step can be included into the washing procedure after each washing step regardless of the type of washing solution used. In another embodiment, a centrifuge step is included after each expansion and contraction step. As illustrated in Table 3 below, employment of a centrifuge step can increase the efficiency of the purification process and enhance the swelling rate of the purified dried SPH. In particular, the same SPH batch from Table 3 was cut into three samples, each subjected to different purification steps as shown in Table 3.

TABLE 3

| | | | Expansion/ |
|---|---|---|---|
| | | | Contraction |
| | | Expansion/ | assisted with |
| | Standard Washing | Contraction | centrifuge |
| Analyte | μg/g of SPH | μg/g of SPH | μg/g of SPH |
| Acrylic monomer | <2.62 | <2.62 | <2.62 |
| Acrylate ester | <24.3 | <24.3 | <24.3 |
| monomer | | | |
| Acrylate salt | <2.46 | <2.46 | <2.46 |
| monomer | | | |
| Stabilizer | <25.3 | <25.3 | <25.3 |
| Diacrylate | <9.48 | <9.48 | <9.48 |
| Crosslinker | | | |
| Rate Swelling* | >4 min | 100-120 sec | 20-40 sec |

*Time by which the SPH reaches its full swelling capacity

The very-pure SPH and methods of purifying according to the present invention can be prepared as shown in the following examples and description thereof, as well as relevant published literature procedures that can be used by one skilled in the art. The following examples serve to better illustrate, but not limit, multiple embodiments of the invention.

### EXAMPLE 1

### A Very-Pure Superporous Hydrogel Based on a Potassium Salt of Sulfopropyl Acrylate

To prepare the hydrogel, an aqueous mixture of sulfopropyl acrylate (3 g), poly (ethylene glycol) diacrylate (0.05 ml), F127 foam stabilizer (0.08 g) citric acid (0.167 g), tetramethyl ethylenediamine (0.08 ml), ammonium persulfate (0.04 g) and sodium bicarbonate (0.3 g) were polymerized according to the process of the present invention. In order to establish a washing procedure, the swelling profile of the synthesized hydrogel was obtained in multiple water/ethanol solutions having differing concentrations as identified in Table 4 below.

TABLE 4

| | Region 1 | Region 2 | Region 3 |
|---|---|---|---|
| Ethanol Cont. v/v % | 0-55 | 55-70 | 70-100 |
| Dimensional Increase | 7-5x | 5-2x | 1.7-1x |

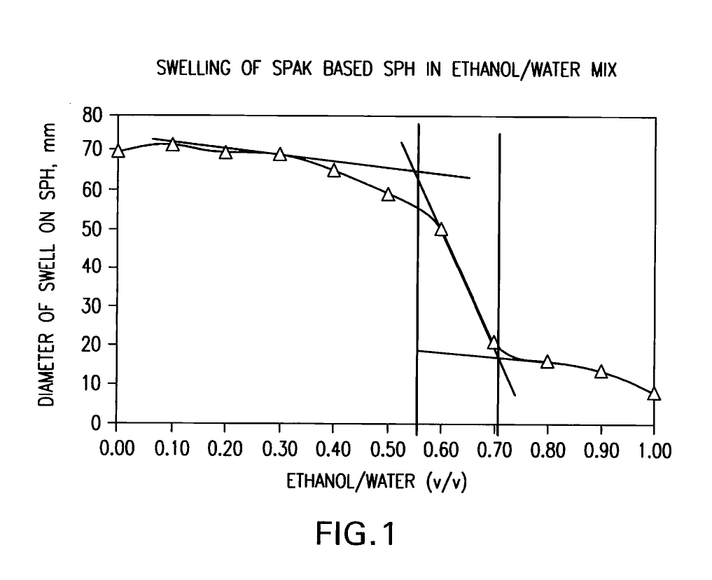

Ethanol content v/v % indicates the volume of ethanol per 100 volumes of combined ethanol and water. FIG. 1 illustrates a graphical representation of the swelling data (as measured in diameter of swollen SPH) of 3-sulfopropyl acrylate potassium salt (SPAK) for various water/ethanol ratios. The SPH swelling was measured dimensionally. In other words, the diameter ratio of the swollen to non-swollen SPH was used as the swelling parameter. Samples of a same batch of SPH were placed into different swelling media and the SPH diameter was measured after the SPHs reached their equilibrium swelling capacities. As data show, a given SPH swells differently in aqueous solutions containing varying amounts of alcohol. Three distinct regions (see vertical lines at ˜55 v/v % and ˜72 v/v %) are identified on the graph, as outlined in Table 4 above.

Region 1 is associated with maximum undesirable swelling in the washing solution. In this region, the swollen gel loses its integrity and starts to disintegrate or erode under any external pressure. Region 3 is associated with almost no swelling. In the absence of swelling, the chance of removing impurities is minimized owing to the limited diffusion coefficients and surface area. However, this region is desirable for dehydrating the gel. Within Region 2, the swelling capacity of the gel sharply decreases with an increase in alcohol concentration. This feature was used to determine different washing steps for this typical high swelling superporous hydrogel. A total of four washing and purification steps were assigned to this gel, one from Region 2 and the rest selected from the Region 3 in which washing and dehydration takes place at the same time. The four wash steps are outlined below.

1-1 Wash the gels in 65 v/v % aqueous ethanol solution

1-2 Decant the solution

2-1 Wash the gels recovered from step 1-2 in 75 v/v % aqueous ethanol solution

2-2 Decant the solution

3-1 Wash the gels recovered from step 2-2 in 90 v/v % aqueous ethanol solution

3-2 Decant the solution

4-1 Wash the gels recovered from step 3-2 in pure alcohol

4-2 Decant the solution

The dehydrated superporous hydrogels taken from step 4-2 were dried out in the mechanical oven at 40° C. overnight. Using validated HPLC methods, the synthetic impurities of the final dried superporous hydrogel were found as shown in Table 5 below.

TABLE 5

| | Concentration | Limit of Quantification |
|---|---|---|
| Analyte | μg/g of SPH | μg/g of SPH |
| Acrylic monomer | BLQ* | 2.62 |
| Sulfopropyl | 4.05 | 2.46 |
| acrylate | | |
| Monomethyl ether | BLQ | 2.22 |
| of Hydroquinone | | |
| Diacrylate | BLQ | 9.48 |
| Crosslinker | | |

*BLQ: Below Limit of Quantification

### EXAMPLE 2

### A Very-Pure Superporous Hydrogel Based on a Potassium Salt of Sulfopropyl Acrylate and an Acrylate Ester

To prepare the hydrogel, an aqueous mixture of sulfopropyl acrylate (0.55 g), hydroxyethyl acrylate (1.24 ml), poly (ethylene glycol) diacrylate (0.05 ml), F127 foam stabilizer (0.08 g), citric acid (0.167 g), tetramethyl ethylenediamine (0.08 ml), ammonium persulfate (0.04 g) and sodium bicarbonate (0.25 g) were polymerized. In order to establish a washing procedure, swelling profile of the synthesized hydrogel was obtained in different water/ethanol mixes as described in Example 2.

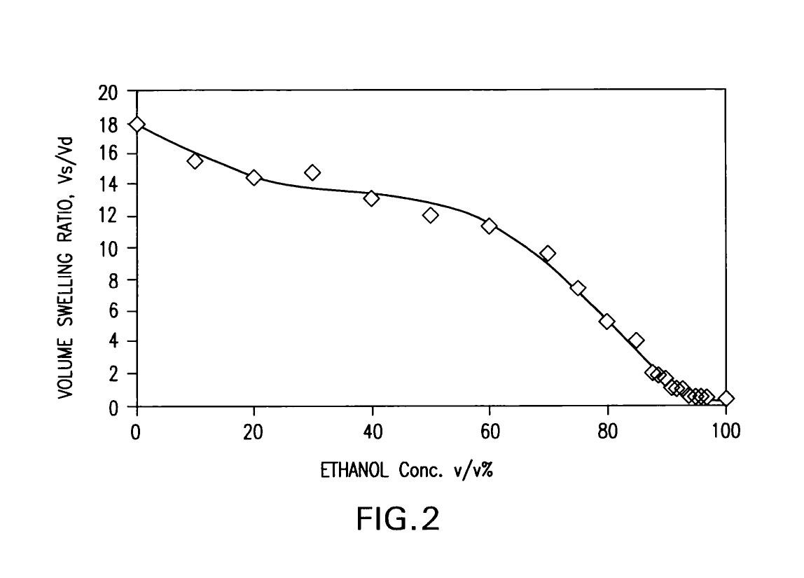

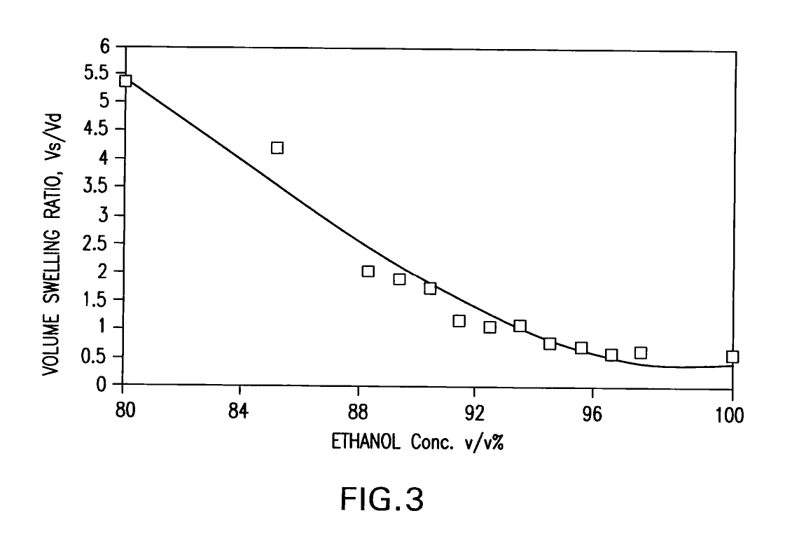

In order to maintain mechanical integrity, the superporous hydrogel should preferably not swell to larger than 1.2 times of its original dimension. To establish a washing procedure, four washing steps were selected based on swelling data in FIGS. 2 and 3. The SPH swelling was measured volumetrically. In other words, the volume ratio of the swollen to non-swollen SPH was used as the swelling parameter. Samples of a same batch of SPH were placed into different swelling media and the SPH volume was measured after the SPHs reached their equilibrium swelling capacities. As data show, a given SPH swells differently in aqueous solutions containing varying amounts of alcohol, but the swelling variation with alcohol concentration is smoother compared to the swelling data in Example 1. Although two different methods have been used to measure the swelling capacity, the overall swelling profile will be similar for both swelling methods. Since SPHs in Example 1 swell significantly in the region 1 of the swelling profile, volumetric measurement was not very feasible and reliable. For this reason, dimensional swelling was adopted. The synthesized gels were washed in washing solutions as follows.

1-1 Wash the gels in 89 v/v % aqueous ethanol solution

1-2 Decant the solution

2-1 Wash the gels recovered from step 1-2 in 94 v/v % aqueous ethanol solution

2-2 Decant the solution

3-1 Wash the gels recovered from step 2-2 in 98 v/v % aqueous ethanol solution

3-2 Decant the solution

4-1 Wash the gels recovered from step 3-2 in pure alcohol

4-2 Decant the solution

The dehydrated superporous hydrogels taken from step 4-2 were dried out in a mechanical oven at 40° C. overnight. Analytical results on extractable monomer(s), crosslinker and inhibitor are illustrated below in Table 6.

TABLE 6

| | Concentration | Limit of Quantification |
|---|---|---|
| Analyte | (μg/g of SPH) | (μg/g of SPH) |
| Acylate Ester | 21.6 | 2.54 |
| Acrylate Salt | 2.61 | 2.46 |
| Inhibitor | BLQ | 25.3 |
| Diacrylate | BLQ | 9.48 |
| Crosslinker | | |

μg/g = one microgram of impurity per one gram of dry SPH

The data of Table 6 illustrates that SPH of Example 2 contain very low levels of impurities even when modifying the monomer composition. This demonstrates that one embodiment of the washing procedures of the invention are capable of reducing impurity levels down to 2-25 ppm (equivalent of μg/g).

### EXAMPLE 3

### A Very Pure High Swelling Synthetic Swelling Agent Based on Acrylate Ester and Acrylate Salt

To prepare the hydrogel, an aqueous mixture of sulfopropyl acrylate (0.55 g) hydroxyethyl acrylate (1.24 ml), poly (ethylene glycol) diacrylate (0.05 ml). F127 foam stabilizer (0.08 g), acetic acid (0.15 ml), tetramethyl ethylenediamine (0.08 ml), ammonium persulfate (0.04 g) and sodium bicarbonate (0.25 g) were polymerized. The synthesized SPH were washed and purified according to the following procedure:

1-1 Wash the gels in 85 wt % aqueous ethanol solution (gel expansion) for 1 hour

1-2 Decant the solution and centrifuge the gels

1-3 Wash the centrifuged gels in pure alcohol (gel contraction) for 0.5 hour

1-4 Decant the solution and centrifuge the gels

2-1 Wash the gels in 86 wt % aqueous ethanol solution (gel expansion) for 1 hour

2-2 Decant the solution and centrifuge the gels

2-3 Wash the centrifuged gels in pure alcohol (gel contraction) for 0.5 hour

2-4 Decant the solution and centrifuge the gels

3-1 Wash the gels in 87 wt % aqueous ethanol solution (gel expansion) for 1 hour

3-2 Decant the solution and centrifuge the gels

3-3 Wash the centrifuged gels in pure alcohol (gel contraction) for 0.5 hour

3-4 Decant the solution and Centrifuge the gels

4-1 Wash the centrifuged gels in pure alcohol (gel contraction) for 1 hour

4-2 Decant the solution and Centrifuge the gels

The centrifuged gels were spread onto a plastic screen, transferred to a mechanical oven and dried out at 45° C. overnight. The amounts of residual monomer(s), crosslinker and the inhibitor are illustrated below in Table 7.

TABLE 7

| | Concentration |
|---|---|
| Analyte | (μg/g of SPH) |
| Acrylic monomer | <2.62 |
| Acrylate ester monomer | <24.3 |
| Acrylate salt monomer | <2.46 |
| Diacrylate Crosslinker | <9.48 |
| Inhibitor | <25.3 |

The data in Table 7 illustrates that very-pure SPH can be achieved using the processes of the invention.

### EXAMPLE 4

The swelling properties of the purified SPH are characterized as the weight swelling ratio and the core disappearance time. The weight swelling ratio is the weight of the swollen gel to that of the dry gel. The SPH core disappearance is the time by which the white core or the white center of the SPH sample swells and hence disappears.

A single SPH batch of Example 3 was prepared in accordance with the invention dissected into five samples and each sample purified in the same manner in accordance with the purification processes of the invention. The results for five samples are illustrated below in Table 8.

TABLE 8

| | SPH Sample | | | | |
|---|---|---|---|---|---|
| | SPH-1 | SPH-2 | SPH-3 | SPH-4 | SPH-5 |
| Dry Weight, g | 0.49 | 0.51 | 0.53 | 0.52 | 0.51 |
| SPH core | 22 | 22 | 30 | 23 | 23 |
| disappearance | | | | | |
| time (seconds) | | | | | |
| Swelling after | 94.4 | 96.4 | 97.3 | 95.9 | 95.5 |
| 30 minutes | | | | | |
| Weight swelling | 193 | 189 | 184 | 184 | 187 |
| ratio, g/g | | | | | |

| | |
|---|---|
| Mean core | 24 |
| disappearance | |
| time (seconds) | |
| Mean weight | 187 |
| swelling ratio, | |
| g/g | |

Differences in swelling data for the individual samples show statistically insignificant deviation from each other and the sample average (mean). This demonstrates that the original SPH batch is very homogeneous. Theoretically, all 5 samples have same concentration of LG/HG monomers as they have been taken from a same batch. Taken together, data from Tables 7 and 8 demonstrate that a very pure (Table 7) high swelling (Table 8) SPH can be produced according to the embodiments of the present invention. The dehydrated, very-pure superporous hydrogels of the present invention rapidly swell to a relatively large size when placed in contact with aqueous fluids; yet remain mechanically strong in their swollen state. Taking advantage of these properties, these hydrogels can be useful as drug delivery systems (DDSs), as described by Park, et al., in Biodegradable Hydrogels for Drug Delivery, 1993, Technomic Pub. Co. or in Hydrogels and Biodegradable Polymers for Bioapplications (ACS Symposium Series, 627), 1996, Eds., Ottenbrite, et al., American Chemical Society.

Drug delivery can involve implanting a controlled release system within a matrix of a dehydrated superporous hydrogel of the invention. This, in turn, would be contained in a capsule (e.g., a gelatin capsule) or similar housing system that can be eroded by the acidic conditions in the stomach. The gastric retention of superporous hydrogels is based on their fast and high swelling properties. Once a superporous hydrogel of the invention is exposed to gastric fluid, it rapidly swells to its maximum swelling capacity, typically in less than ten minutes. For their use in humans, superporous hydrogels that swell to a diameter of greater than 2 cm at low pH conditions are desirable as they are then unable to pass through the pylorus sphincter, ensuring prolonged residence in the stomach and better absorption of the drug through the upper GI tract.

In addition to drug delivery, the hydrogels of the invention can have a variety of applications including, for example, as a diet aid in food industry, as a swelling agent in pharmaceutical industry, tissue engineering, vascular surgery (e.g., angioplasty) and drainage (e.g., from the kidney). Devices prepared using hydrogels of the invention can include, but are not limited to, vascular grafts, stents, catheters, cannulas, plugs, constrictors, tissue scaffolds, and tissue or biological encapsulants, and the like. Disclosure and examples for additional application and utility of the very-pure SPH of the present invention are described in U.S. Pat. No. 7,056,957.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall with the spirit and broad scope of the appended claims. All patent applications, patents, and other publications cited herein are incorporated by reference in their entirety.

What we claim is:

1. A method of producing a very-pure and high swelling superporous hydrogel comprising the steps of:
(a) preparing a superporous hydrogel by reacting a mixture comprising at least one high-glass transition temperature (Tg) ethylenically unsaturated monomer (HG) and at least one low-glass transition temperature (Tg) ethylenically unsaturated monomer (LG);
(b) evenly integrating a foaming agent within the superporous hydrogel reacting mixture of step (a) using a integration means for creating a homogeneous mixture of the foaming agent within the superporous hydrogel reacting mixture;
(c) washing the superporous hydrogel foam of step (b) one or more times in a washing solution comprising a solvent and a non-solvent to remove impurities from the hydrogel of step (b); and
(d) drying the washed superporous hydrogel of step (c) to form a very-pure superporous hydrogel.

2. The method of claim 1 further comprising the step of mechanically separating the superporous hydrogel of step (c) after a first wash.

3. The method of claim 2 wherein said mechanical separating step is rubbing.

4. The method of claim 1 further comprising the step of removing the washing solution from the superporous hydrogel after each wash performed in step (c) prior to initiating another washing step thereof.

5. The method of claim 1 wherein the superporous hydrogel reacting mixture further comprises one or more components selected from the group consisting of a solvent, a foam stabilizer, a foaming aid, a cross linker, an initiator, and combinations thereof.

6. The method of claim 4 wherein said removing step of the washing solution from the superporous hydrogel further comprises the step of using at least one of a mechanical centrifuge, filtration or decantation.

7. The method of claim 1 wherein each wash of step (c) utilizes a washing solution further comprising different ratios of solvent to non-solvent to cause expansion and contraction of the superporous hydrogel with each wash.

8. The method of claim 1 wherein step (c) further comprises transferring the superporous hydrogel into a solution of substantially pure non-solvent (e.g., ethanol) after a wash in the washing solution.

9. The method of claim 1 wherein said integration means is a powder gun.

10. The method of claim 1 wherein steps (a) and (b) are performed simultaneously.

11. The method of claim 1, wherein said at least one high-glass transition temperature (Tg) ethylenically unsaturated monomer is selected from the group consisting of acrylic acid (AA), methacrylic acid, (meth)acrylic salts, acrylamide (AM), N-isopropylacrylamide (NIPAM), methacrylamide, itaconic acid, potassium 3-sulfopropyl acrylate (SPAK), potassium 3-sulfopropyl methacrylate (SPMAK), hydroxyethyl methacrylate (HEMA), hydroxypropyl methacrylate (HPMA) and stearyl methacrylate.

12. The method of claim 1, wherein said at least one low-glass transition temperature (Tg) ethylenically unsaturated monomer is selected from the group consisting of N,N-dimethylaminoethyl acrylate, 2-hydroxyethyl acrylate (HEA), 2-hydroxypropyl acrylate (HPA), butanediol monoacrylate, ethyl acrylate, butyl acrylate, butyl methacrylate, isodecyl methacrylate and lauryl methacrylate.

13. The method of claim 1 wherein the ratio of the one or more low-glass transition temperature (Tg) ethylenically unsaturated monomers to the one or more high-glass transition temperature (Tg) ethylenically unsaturated monomers is about 1:10 to 10:1.

14. The method of claim 13 wherein the low and the high Tg monomers are 2-hydroxyethyl a acrylate and potassium salt of 3-sulfopropyl acrylate respectively.

15. The method of claim 7, wherein a solvent and non-solvent composition characterized by a medium to high solvent content contains greater than 10 v/v % solvent, and a solvent and non-solvent composition characterized by medium to low solvent content contains less than 10 v/v % solvent.

16. A method for prolonging retention of a pharmaceutical agent by gastric retention comprising administering to a patient a very-pure superporous hydrogel prepared by the method of claim 1 and further comprising a pharmaceutical agent, wherein said hydrogel swells upon entering the stomach of the patient and prevents emptying of said pharmaceutical agent from stomach for at least one hour.

17. The method of claim 16 wherein the pharmaceutical agent is a drug or a nutritional supplement.

18. A method for controlling the appetite of a patient comprising administering a very-pure superporous hydrogel prepared by the method of claim 1, wherein said hydrogel swells upon entering the stomach of the patient.

19. A method for controlling the appetite of a patient comprising administering a formulated very-pure superporous hydrogel prepared by the method of claim 1 wherein the hydrogel swells upon entering the stomach of the patient.

20. The method of claim 19, in which a formulated very pure superporous hydrogel further comprises nutrition, vitamin, an acid-neutralizing agent and buffer agent.
